(19) United States
(12) Patent Application Publication
(10) Pub. No.: US 2005/0177194 A1
(43) Pub. Date: Aug. 11, 2005

(54) Heart monitoring and stimulating device, a system including such a device and use of the system

(76) Inventor: Anders Bjorling, Jarfalla (SE)

Correspondence Address: SCHIFF HARDIN, LLP;PATENT DEPARTMENT, 6600 SEARS TOWER, CHICAGO, IL, 60606-6473, US

(21) Appl. No.: 10/513,769

(22) PCT Filed: Feb. 14, 2003
(86) PCT No.: PCT/SE03/00245

(30) Foreign Application Priority Data
Jun. 14, 2002 (SE) 0201822-4

(51) Int. Cl. A61N 1/362

(52) U.S. Cl. 607/9

(57) ABSTRACT

An implantable heart monitoring and stimulating device has a control circuit and a memory, the control circuit receiving signals representing detected cardiac events respectively from the right and left ventricles of a heart. The control circuit controls a pulse generator to transmit stimulation pulses to the right and left ventricles as well. The control circuit determines the time, within a heart cycle, between a first event related to the right ventricle and a second event related to the left ventricle, or vice versa. The determined time is stored in the memory. The determination and storage of the aforementioned time are repeated at a number of occasions.

### BACKGROUND OF THE INVENTION

1. Field of the Invention

The present invention relates to an implantable heart monitoring and stimulating device of the type having a control circuit and a memory connected to the control circuit. The control circuit is adapted to be connected to at least a first sensing member and a second sensing member. The first sensing member is adapted for sensing cardiac events related to the right ventricle of the heart and the second sensing member is adapted for sensing cardiac events related to the left ventricle of the heart. The control circuit receives signals from said first and second sensing members such that cardiac events of said right and left ventricles are detectable by the control circuit. The control circuit is also adapted to be connected to at least a first stimulation member and a second stimulation member. The first stimulation member is adapted for electrically stimulating the right ventricle of the heart and the second stimulation member is adapted for electrically stimulating the left ventricle of the heart. The control circuit transmits stimulation pulses to the first and second stimulation members in order to stimulate the right and left ventricles.

The present invention also relates to a system including such a device and to a method for monitoring the performance of a heart of a human or animal being as well for stimulating the heart.

2. Description of the Prior Art

Many different implantable devices of the above general type for monitoring and stimulating a heart are known. The devices normally are able to sense the electrical activity of the heart. It is also known to sense other physiological parameters, such as blood pressure, oxygen level etc. Some devices are also able to sense the activity level of the human or animal being into which the device is implanted. With such a device it is possible, for example, to detect whether the person or animal in question is exercising or resting. Some implantable devices are able to deliver stimulation pulses to both the left and right ventricles of the heart, and sometimes also to the left and right atria.

U.S. Pat. No. 5,720,768 describes different possible electrode positions in order to stimulate or sense the different chambers of the heart.

U.S. Pat. No. 6,070,100 describes that electrodes may be positioned in both the left and the right atrium as well as in the left and the right ventricles.

U.S. Pat. No. 5,21,098 describes a cardiac stimulator with an oxygen saturation sensor positioned in the coronary sinus of the heart. This device is also able to sense the blood pressure and the electrical activity of the heart.

U.S. Pat. No. 5,199,428 describes a device for detecting myocardial ischemia. A pH sensor or an oxygen saturation sensor may be positioned in the coronary sinus.

U.S. Pat. No. 6,236,873 describes an electrochemical sensor for measuring the oxygen content in blood.

U.S. Pat. No. 4,202,339 describes a sensor for measuring the oxygen saturation level in blood.

U.S. Pat. No. 4,453,537 describes a device for sensing, inter alia, the carbon dioxide content in the blood.

U.S. Pat. No. 5,592,170 describes a fiber optic sensor for sensing the nitric oxide content in blood.

It is also known to communicate with an implanted heart monitoring/stimulating device in a wireless manner, i.e. with so-called telemetry. This can be done by inductive communication or via radio waves. With telemetry it is possible, for example, to obtain information about the status of an implanted device. It is also known to input new information into the device by means of such telemetry.

### SUMMARY OF THE INVENTION

An object of the invention is to provide an implantable heart monitoring and stimulating device of the type initially described wherein changes in the heart condition can be monitored in a suitable manner. A further object is to provide such a device in which the condition of the heart of a patient who suffers from cardiomyopathy can be monitored. A still further object is to provide such a device in which the condition of the heart of a patient suffering from a bundle branch block can be monitored in an efficient manner. Still another object is to provide such a device in which the monitored result concerning the heart condition is easy to understand for, for example, a physician.

The above objects are achieved by an implantable heart monitoring device of the type initially described, and wherein the control circuit also (a) determines the time, within a heart cycle, between a first event related to the right ventricle and a second event related to the left ventricle, or vice versa, stores the determined time in the memory, and wherein the control circuit performs (a) and (b) at a number of occasions and stores the corresponding determined times in the memory.

By determining these times and storing these times in the memory, the memory contains information concerning the heart condition at the different occasions. This information may then be used to analyze how the heart condition has changed over time. The invention is useful for example, in connection with a patient suffering from a dilated cardiomyopathy. In particular, left bundle branch block in many cases can lead to a dilated cardiomyopathy, whereby the patient's left ventricle will be enlarged. This will affect the cardiac performance of the patient. The enlargement of the left ventricle may lead to a slower contraction of the left ventricle. This makes the burden on the left ventricle higher, which may lead to a further enlargement of this ventricle. The underlying cause of this condition, for example caused by left bundle branch block, is that the left and right ventricles are not synchronized with each other. The synchronization may be improved by using an implanted biventricular pacemaker. When the heart condition is improved, such that the left and right ventricles will be more synchronized, the size of the left ventricle is likely to decrease. This may improve the conduction properties of the heart. The delay between events of the right and left ventricles will therefore become smaller. With the present invention it is therefore possible to analyze whether the heart condition is improving or getting worse with regard to the described condition.

It should be noted that at each occasion, the aforementioned time may be determined once and stored. It is also possible to determine the time with several repetitions at the aforementioned occasion and to determine a relationship between the different determined times (for example an average value of the determined times) at the occasion, and to store the relationship in the memory.

In a preferred embodiment of the invention, the aforementioned first event is a sensed R-wave of one ventricle and the second event is a corresponding sensed R-wave in the other ventricle of the heart. The control circuit causes no stimulation pulses to be delivered to the stimulation members at least for the heart cycles when the time is determined between the sensed R-waves.

According to another embodiment, the first event is the delivery of a stimulation pulse to one ventricle and such that the second event is a sensed R-wave in the other ventricle. The control circuit causes no stimulation pulse to be delivered to the ventricle in which the R-wave is sensed, at least for the heart cycle when the time is determined.

It should be noted that the above described manners of determining the time may also be combined such that both the time between two R-waves and the time between a stimulation pulse and the R-wave are determined.

All these determined times relate to the magnitude of the delay that occurs between events in the left and the right ventricles. Therefore, by monitoring how these times differ between the different occasions, the change of the heart condition can be monitored.

According to a further embodiment, the control circuit senses the activity level of a human or animal being in which the device is implanted. The control circuit selects the occasions to carry out the determination of the time when the activity level is at a predetermined level or within a predetermined range. Preferably, the activity level during these occasions represents, a low level of activity. The determination of the time, for example, may be carried out when the human or animal being is asleep. By always determining the times at approximately the same level of activity, an accurate indication of the change of the heart condition is obtained.

The control circuit, for example, may operate so that the time or times are determined at least once a month, preferably at least once a week. The control circuit preferably operates so that the time or times are not determined too often, for example not more often than each hour or preferably not more often than once a day. With such intervals between these occasions, a sufficient indication of the change of the heart condition is obtained without using too much space is the memory. It should be noted, however, that it is possible for the time or times to be determined more often than once each hour.

According to another preferred embodiment, the device and the control circuit detect at least one pacing or physiological parameter such as the pacing rate, blood pressure, blood oxygen content, metabolic substance in the blood or the activity level of the human or animal being in which the device is implanted, and the control circuit determines the aforementioned times at different values of this parameter. The times may be determined, for example, at each of a higher pacing rate and at a lower pacing rate. The different determined values may be useful in further analyzing the heart condition. Similar determinations may be done for other pacing or physiological parameters.

The above object also is achieved in accordance with the invention by an implantable heart monitoring and stimulating system comprising a device according to any of the above embodiments and a first lead and a second lead connected to the device. The first sensing member is disposed on the first lead and the second sensing member is disposed on the second lead. Preferably, also the first stimulation member is disposed on the first lead and the second stimulation member is arranged on the second lead. In a preferred embodiment, the same member may be used for the first sensing member and the first stimulation member. Similarly, the same member may be used for the second stimulation member and the second sensing member. Such a device may be implanted in a human or animal being. With such a device the above-mentioned advantages are achieved.

Tape

A preferred manner of using the system includes transferring the values of the determined times that are stored in the memory to an external apparatus, which does not form part of the implanted system. For example, the determined values may be transferred by telemetry, such as is known to those skilled in the art. Using the external apparatus, it is possible to analyze the stored values in order to find how the heart condition has changed over time.

According to a preferred use, a graph that represents the change of the heart condition of the human or animal being is produced. The system is particularly useful in order to monitor the change of the heart condition for a human or animal being suffering from cardiomyopathy, for example caused by a bundle branch block.

### BRIEF DESCRIPTION OF THE DRAWINGS

FIG. 1 schematically illustrates a heart monitoring system with a heart monitoring device connected to leads with sensing and stimulation members positioned in a heart.

FIG. 2 schematically illustrates, on a time scale, sensed R-waves in the two ventricles of a heart.

FIG. 3 schematically illustrates, on a time scale, a delivered stimulation pulse to the right ventricle and a sensed R-wave of the left ventricle.

FIG. 4 schematically illustrates, on a time scaleU, a stimulation pulse delivered to the left ventricle and a sensed R-wave of the right ventricle.

FIG. 5 a flow chart of the functioning of a heart monitoring system according to an embodiment of the invention as well as a method for using such a system.

FIG. 6 schematically illustrates how a time determined at different occasions in accordance with the invention may change over time.

### DESCRIPTION OF PREFERRED EMBODIMENTS

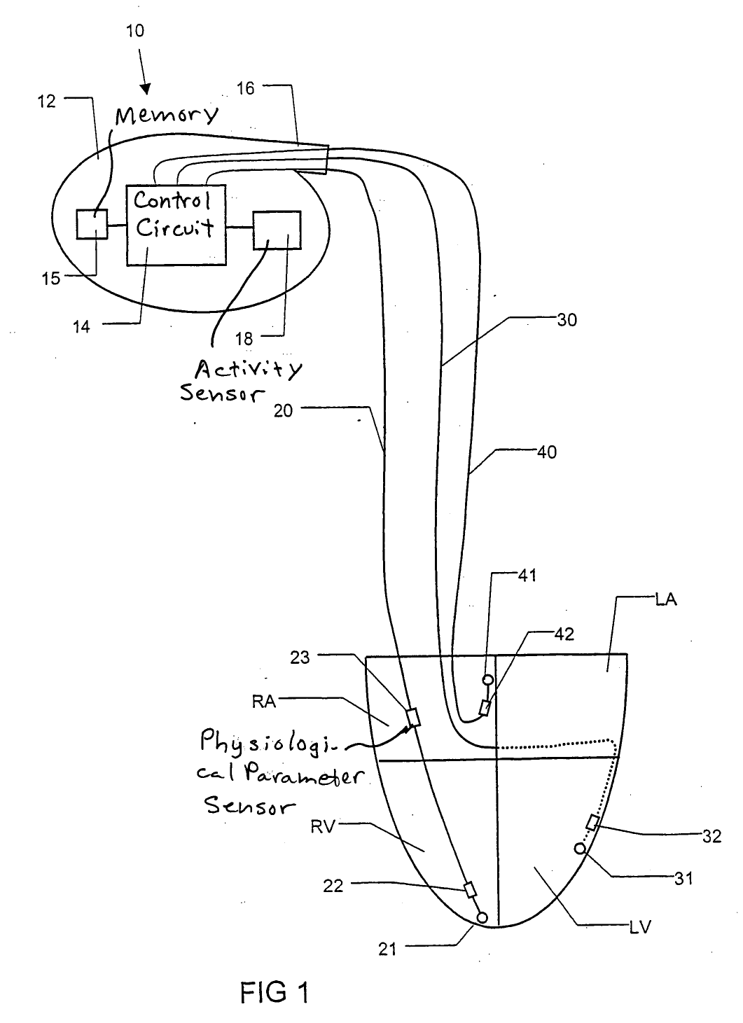

FIG. 1 schematically shows an implantable heart monitoring and stimulating system according to the invention. This system includes an implantable heart monitoring and stimulation device 10 according to the invention. The device 10 has a housing 12; inside of which is a control circuit. The device 10 may constitute a pacemaker which is also able to detect electrical signals from a heart. Since such a device (with the exceptions described below) is well known to those skilled in the art, the details of this device need not be described herein. The device has a connector portion 16. Different leads 20, 30, 40 may be connected to the control circuit 14 via the connector portion 16. The device has a memory 15 connected to the control circuit 14. In the memory 15, data may be stored. Furthermore, the device 10 may include an activity sensor 18 to enable sensing of the activity level of a living being into which the device 10 is implanted.

The device 10 may also operate to sense different physiological parameters, such as blood pressure, blood oxygen content, metabolic substances in the blood, or other physiological parameters. Such parameters may be sensed by suitable sensors connected to the device 10. The sensors for example, may be carried on one of the leads 20, 30, 40. One such sensor is indicated by 23.

FIG. 1 shows three leads 20, 30, 40 connected to the device 10. The number of the leads may be more or less than three. The first lead 20 includes a first sensing member including two electrode surfaces 21 and 22 in this embodiment. The electrode member 21 may be a tip electrode and the electrode member 22 may be a ring electrode. The electrode surfaces 21, 22 thus form bipolar electrodes, but it is also possible for the device 10 to be connected to unipolar electrodes. In such an arrangement, the housing 12 usually functions as a second electrode. Devices with unipolar and bipolar electrodes are well known to those skilled in the art and need not be described in more detail herein.

According to a preferred embodiment, the first sensing member 21, 22 also fulfils the function of a first stimulation member 21, 22. This member 21, 22 may thus be used to deliver stimulation pulses as well as to detect the electrical activity of the heart.

FIG. 1 shows schematically a heart with a right atrium RA, a right ventricle RV, a left atrium LA and a left ventricle LV. The first lead 20 is introduced such that the first sensing and stimulation member 21, 22 is positioned in the right ventricle RV.

A second lead 30 includes a second sensing member formed by electrode surfaces 31, 32. This member 31, 32 may also function as a second stimulation member 31, 32. The second sensing and stimulation member 31, 32 is disposed such that it can sense and stimulate the left ventricle LV of the heart. The second lead 30, for example, may be introduced via the right atrium into the coronary sinus such that the member 31, 32 is positioned in the middle or great cardiac vein. Now to introduce the second lead 30 in this manner is known to those skilled in the art as indicated in some of the above-mentioned documents.

FIG. 1 also shows a third lead 40 with a sensing and stimulation member formed by electrode surfaces 41, 42 positioned in the right atrium RA.

The different leads 20, 30, 40 of course include conductors (not shown) in order to conduct signals between the device 10 and the sensing and stimulation members 21, 22, 23, 31, 32, 41, 42. The device 10 together with attached leads 20, 30, 40 thus constitute a system according to the invention.

According to the invention, the control circuit 14 includes a sensing circuit that, via the first sensing member 21, 22, senses cardiac events related to the right ventricle RV. Furthermore, the control circuit 14 includes a sensing circuit that, via the second sensing member 31, 32, senses cardiac events related to the left ventricle LV. Furthermore, the control circuit 14 includes pacing circuits (pulse generators) to deliver stimulation pulses to the first stimulation member 21, 22 and the second stimulation member 31, 32 in order to stimulate the right ventricle RV and the left LV ventricle, respectively. The control circuit 14 is also
a) determines the time T1, T2, T3, within a heart cycle, between a first event related to the right ventricle RV and a second event related to the left ventricle LV, or vice versa,
b) stores the determined time T1, T2, T3 in the memory 15, and
c) performs the steps a) and b) at a number of occasions and stores the corresponding determined times T1, T2, T3 in the memory 15.

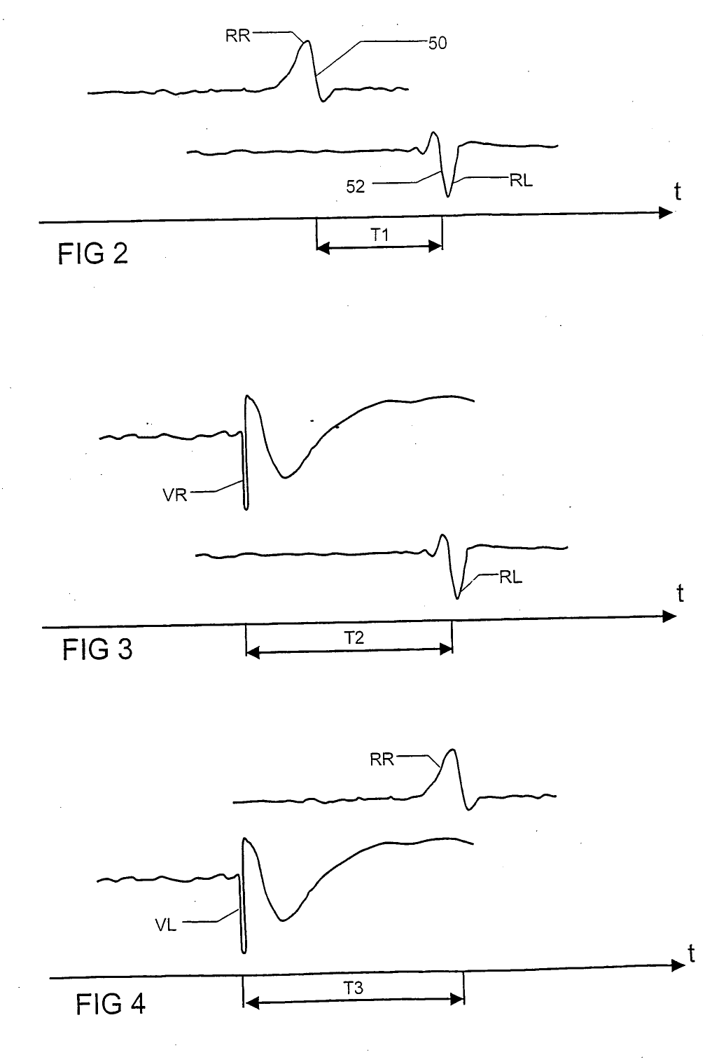

With reference to FIGS. 2, 3 and 4 the significance of these events will be explained. The upper graph in each of these figures relates to the right ventricle and the lower graph relates to the left ventricle LV. These graphs are shown on a time scale t in order to see when the different events occur in time.

FIG. 2 shows a detected R-wave RR of the right ventricle RV. This wave RR may thus be detected with a help of the first sensing member 21, 22. FIG. 2 also shows a detected R-wave RL of the left ventricle LV. This wave RL may thus be detected by the second sensing member 31, 32. The time between the waves RR and RL is indicated by T1. In a healthy heart the time T1 is often less than 20 ms. The time T1 in a healthy heart depends, inter alia, on where in relation to the ventricles the sensing is done. However, in a heart suffering from a bundle branch block, for example, the time T1 is essentially longer. The occurrence of each wave RR and RL may be defined in different manners. For example, it is possible to determine the time between the peaks of each wave RR and RL. Another possibility is to detect the maximum negative derivative of each wave RR, RL. The point of the maximum negative derivative of the wave RR is indicated by 50. The point of the maximum negative derivative of the wave RL is indicated by 52. According to an embodiment of the invention, the control circuit 14 determines the time T1 and stores this time T1 in the memory 15. The control circuit 14 carries out different determinations of T1 at different occasions and stores the different determined values of T1 in the memory 15. It is thereby possible to see how T1 has changed over time. During the heart cycles when T1 is determined, the control circuit 14 is arranged such that no stimulation pulses are delivered to the first stimulation member 21, 22 and the second stimulation members 31, 32.

The upper graph of FIG. 3 shows a stimulation pulse VR delivered to the right ventricle RV with the first stimulation member 21, 22. The lower graph in FIG. 3 shows a detected R-wave RL of the left ventricle LV. This wave RL may thus be detected by the second sensing member 31, 32. The control circuit 14 determines the time T2 between the stimulation pulse VR and the R-wave RL. Also in this case the occurrence of the R-wave RL may be defined in different, ways, for example as the maximum positive or negative derivative. Since the stimulation pulse VR is delivered by the control circuit 14, the delivery of this pulse VR may trigger a time counter to start measuring the time T2. The occurrence of the R-wave RL then determines the end of the time interval T2. The control circuit 14 is arranged such that during the heart cycles when the time T2 is determined, no stimulation pulse is delivered to the left ventricle LV, i.e. no stimulation pulse is delivered to the second stimulation member 31, 32. The control circuit 14 determines the time T2 at different occasions and stores the determined values of T2 in the memory 15.

The lower graph in FIG. 4 shows a stimulation pulse VL delivered to the left ventricle LV with the help of the second stimulation member 31, 32. The upper graph in this figure shows a detected R-wave RR originating from the right ventricle RV. This wave RR may be detected by the first sensing member 21, 22. The control circuit 14 determines the time T3 between the delivered stimulation pulse VL and the sensed R-wave RR. This can be done in an analogous manner to that described above. The control circuit 14 causes no stimulation pulse to be delivered to the right ventricle RV during the heart cycles when the time T3 is determined. The control circuit 14 determines the time T3 at different occasions and the different values of T3 are stored in the memory 15.

According to the invention, at each occasion one of the values T1, T2, T3 or any two of the values T1, T2, T3 are determined. According to one embodiment of the invention, the control circuit 14, at each occasion, determines all three times T1, T2, T3 in accordance with the above description. It should be noted that as used herein “occasion” can encompass a short time span within which the different values T1, T2, T3 are being determined. It should also be noted that it is possible that, at each “occasion,” several measures of the time in question (for example T1) are done and an average value of these measures is stored as the determined time T1. If two or more of the values T1, T2 and T3 are being determined at an occasion, it is possible to store each determined value T1, T2, T3 separately from each other in the memory 15. Alternatively, it is also possible to store a combination (for example an average value) of these values T1, T2 and T3 in the memory 15.

According to a preferred embodiment, the control circuit 14 is arranged such that is receives an indication of the activity level of the human or animal being into which the device 10 is implanted. The control circuit 14 may occasions, at which the times T1, T2, T3 are determined, as being when the activity level is at a predetermined level or with a predetermined range, preferably at a low level of activity. Since all the different occasions are chosen at approximately the same level or activity, a better comparison of the different determined times can be done. The occasions may be chosen, for example, when the human or animal being in question is asleep.

The control circuit 14 preferably operates so that the occasions occur (i.e. the times are determined and stored) at regular intervals. For example, this can be done at least once a month, preferably at least once a week. In order to save space in the memory 15, the occasions should not occur too frequently, for example not more often than each hour, and preferably not more often than each day. The determination of the times thus may be done once a day or once a week. It is of course within the scope of the invention for the occasions to occur more often than each hour.

According to one embodiment, the control circuit 14 detects at least one pacing parameter or physiological parameter, for example the pacing rate, blood pressure, blood oxygen content, or the content of a metabolic substance in the blood or the above mentioned activity level of the human or animal being into which the device is implanted. According to this embodiment, the control circuit 14 determines the time T1, T2, T3 at different values of the detected parameter and stores these times and measured values in the memory 15. When analyzing the content in the memory 15, it can be seen how the determined times T1, T2, T3 vary dependent on the mentioned parameter.

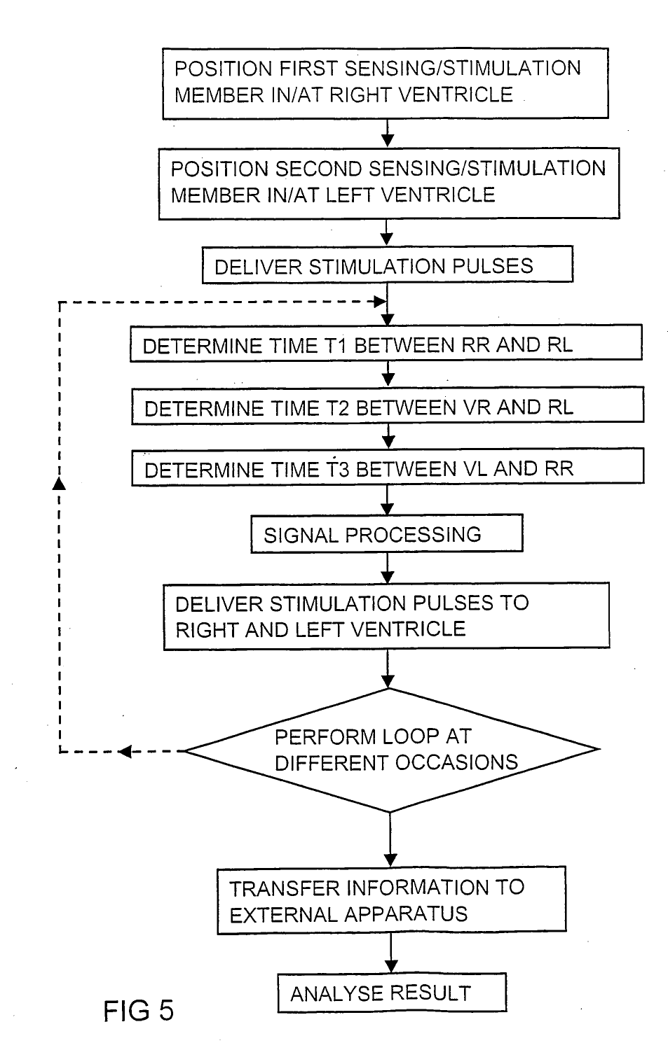

As described above, the invention also concerns an implantable heart monitoring and stimulating system including the device 10 and at least a first 20 and a second 30 lead. The invention also concerns a method for using or operating of such a system for monitoring a heart. With reference to FIG. 5, a use of this system will now be described. At the same time, the function of the device 10 is clear from FIG. 5. According to this use, the device 10 is implanted in a human or animal being and the first sensing/stimulation member 21, 22 is positioned in or at the right ventricle RV. The second sensing/stimulation member 31, 32 is positioned in or at the left ventricle LV. As explained above, this member 31, 32 may for example be positioned in the middle or great cardiac vein. The control circuit 14 is able to deliver stimulation pulses to the right RV and left LV ventricles. Furthermore, at a certain occasion, the time T1 between RR and RL is determined and stored in the memory 15. As mentioned above, no stimulation pulses are delivered during the heart cycles when T1 is being determined. Moreover, according to a preferred embodiment also the time T2 between VR and RL is determined and stored in the memory 15 in accordance with the above description. Furthermore, the time T3 between VL and RR is determined and stored in the manner described above.

According to one embodiment, the control circuit 14 processes the measured times T1, T2, T3, for example in order to form a combined or average value as explained above and to store this value in the memory 15. When the measurements at one occasion have been carried out, the device 10 is normally set to deliver stimulation pulses to the left LV and right RV ventricles. At a later occasion, the times T1, T2, T3 are determined again in accordance with what has been described above.

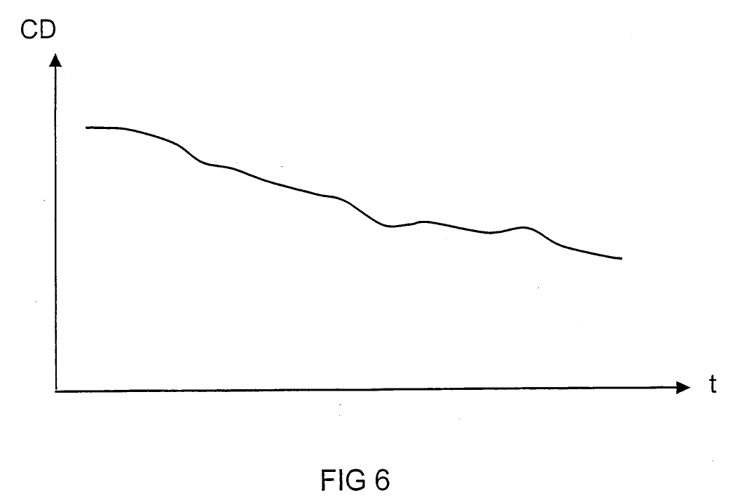

When the loop shown in FIG. 5 has been performed a number of times, the information stored in the memory 15 is transferred to an external apparatus, for example to a computer, which does not form part of the implanted system. This transfer may for example be done in connection with a medical check-up, for example once every half-year or once a year. The result is analyzed in order to see how the heart condition has changed with time. For example, it is possible to form a graph which shows how the determined time varies between the different occasions. FIG. 6 schematically shows such a graph. The Y-axis here shows the conduction delay CD between the right RV and left LV ventricles. This conduction delay CD may be represented by either T1, T2 or T3 or by a value which is a combination of these times. The X-axis shows the time. The shown time may cover, for example, half a year or a year. The graph thus shows whether the conduction between the two ventricles has improved or has gotten worse. In the illustrated example the conduction delay CD has become shorter, which indicates that the heart condition has improved.

The invention is particularly useful in order to monitor the change of the heart condition for a human or animal being, suffering from cardiomyopathy. When the being suffers from a bundle branch block, primarily left bundle branch block, the invention is particularly useful. When the being in question suffers from such a block, the electrical signals in the heart are not conducted in the normal manner but are transferred via the heart tissue between the ventricles. As explained above, the conduction delay CD between the ventricles here depends, inter alia, on whether the Left ventricle LV has been enlarged or not. By monitoring the determined times T1, T2, T3, an indication of the delay in the conduction of the signals between the ventricles is thus obtained. It is thereby possible to monitor how the heart condition changes with time.

Although modifications and changes may be suggested by those skilled in the art, it is the intention of the inventors to embody within the patent warranted hereon all changes and modifications as reasonably and properly come within the scope of their contribution to the art.

1-18. (canceled)

19. An implantable heart monitoring and stimulating device comprising:
a housing adapted for implantation in a subject;
a sensing circuit disposed in said housing and adapted to interact with a right ventricle of a heart in the subject for sensing cardiac events related to the right ventricle, and adapted to interact with a left ventricle of the heart in the subject for sensing cardiac events related to the left ventricle;
a pulse generator adapted to interact with the right ventricle to electrically stimulate the right ventricle and to interact with the left ventricle to electrically stimulate the left ventricle;
a memory in said housing; and
a control circuit disposed in said housing connected to said sensing circuit for receiving signals representing the sensed cardiac events related to the right ventricle and signals representing sensed cardiac events related to the left ventricle therefrom and connected to said pulse generator for controlling stimulation of the right and left ventricle, and having access to said memory, said control circuit determining a time, within a heart cycle, between a first event related to one of said right and left ventricles and a second event related to the other of said right and left ventricles, and storing said determined time in said memory, and performing a plurality of repetitions of determining and storing said time at a plurality of occasions, thereby producing a plurality of stored times in said memory.

20. An implantable heart monitoring and stimulating device as claimed in claim 19 wherein said first event is an occurrence of an R-wave and wherein said second event is an occurrence of an R-wave and wherein said control circuit controls said pulse generator for causing no stimulation pulses to be delivered to said right and left ventricles in a heart cycle wherein said time between said R-waves is being determined.

21. An implantable heart monitoring and stimulating device as claimed in claim 19 wherein said first event is delivery of a stimulation pulse from said pulse generator and wherein said second event is an occurrence of an R-wave sensed by said sensing circuit, and wherein said control circuit controls said pulse generator for causing no stimulation pulse to be delivered to said left ventricle in a heart cycle wherein said time between said stimulation pulse to the left ventricle and the occurrence of said R-wave sensed by said sensing circuit is being determined.

22. An implantable heart monitoring and stimulating device as claimed in claim 19 wherein said first event is delivery of a stimulation pulse to the right ventricle by said pulse generator and wherein said second event is an occurrence of an R-wave from the right ventricle, and wherein said control circuit controls said pulse generator for causing no stimulation pulse to be delivered to the right ventricle in a heart cycle wherein the time between said stimulation pulse to the left ventricle and the occurrence of an R-wave sensed from said right ventricle is being determined.

23. An implantable heart monitoring and stimulating device as claimed in claim 19 comprising an activity sensor adapted to interact the subject to generate a signal indicative of an activity level of the subject, and wherein said control circuit is connected to said activity sensor and determines said time only when the activity level represented by said signal is at a predetermined level.

24. An implantable heart monitoring and stimulating device as claimed in claim 23 wherein said predetermined level is indicative of a low level of activity.

25. An implantable heart monitoring and stimulating device as claimed in claim 19 comprising an activity sensor adapted to interact the subject to generate a signal indicative of an activity level of the subject, and wherein said control circuit is connected to said activity sensor and determines said time only when the activity level represented by said signal is within a predetermined range.

26. An implantable heart monitoring and stimulating device as claimed in claim 25 wherein said predetermined level is indicative of a low range of activity.

27. An implantable heart monitoring and stimulating device as claimed in claim 19 wherein said control circuit selects said occasions to occur at least once a month.

28. An implantable heart monitoring and stimulating device as claimed in claim 19 wherein said control circuit maintains at least one hour between said occasions.

29. An implantable heart monitoring and stimulating device as claimed in claim 19 comprising a detector for at least one parameter selected from the group of pacing rate, blood pressure, blood oxygen content, a metabolic substance in the blood, and an activity level of the subject, and wherein said control circuit determines said time at respective occasions at which said at least one parameter exhibits different values.

30. An implantable heart monitoring and stimulating device comprising:
a housing adapted for implantation in a subject;
a sensing circuit disposed in said housing and adapted to interact with a right ventricle of a heart in the subject for sensing cardiac events related to the right ventricle, and adapted to interact with a left ventricle of the heart in the subject for sensing cardiac events related to the left ventricle;
a pulse generator adapted to interact with the right ventricle to electrically stimulate the right ventricle and to interact with the left ventricle to electrically stimulate the left ventricle;
a memory in said housing; and
a control circuit in said housing for determining a first time between an occurrence of an R-wave in one of said left and right ventricles sensed by said sensing circuit and an occurrence of an R-wave from the other of said first and second ventricles sensed by said sensing circuit, and determines a second time between delivery of a stimulation pulse from the pulse generator to the right ventricle and an occurrence of an R-wave from the left ventricle sensed by said sensing circuit, and determines a third time between delivery of a stimulation pulse from the pulse generator to the left ventricle and an occurrence of an R-wave from the first ventricle sensed by said sensing circuit, and wherein said control circuit controls said pulse generator for causing no stimulation pulses to be delivered by said pulse generator during a heart cycle in which said first, second and third times are being determined, and wherein said control circuit stores said first, second and third times in said memory, and repeats determination and storage of said first, second and third times at a plurality of occasions, thereby producing a plurality of stored first times, a plurality of stored second times and a plurality of stored third times in said memory.

31. An implantable heart monitoring and stimulating system comprising:
a housing adapted for implantation in a subject;
a first lead adapted for implantation in the subject for interacting with the right ventricle of the heart of the subject;
a second lead carrying a second sensing member adapted to interact with the left ventricle of the heart of the subject;
a sensing circuit in said housing connected to said first and second leads for sensing, via said first sensing member, cardiac events related to the right ventricle and for sensing, via said second sensing member, cardiac events related to the left ventricle;
a pulse generator adapted to interact with the right ventricle to electrically stimulate the right ventricle and to interact with the left ventricle to electrically stimulate the left ventricle;
a memory in said housing; and
a control circuit disposed in said housing connected to said sensing circuit for receiving signals representing the sensed cardiac events related to the right ventricle and signals representing sensed cardiac events related to the left ventricle therefrom and connected to said pulse generator for controlling stimulation of the right and left ventricle, and having access to said memory, said control circuit determining a time, within a heart cycle, between a first event related to one of said right and left ventricles and a second event related to the other of said right and left ventricles, and storing said determined time in said memory, and performing a plurality of repetitions of determining and storing said time at a plurality of occasions, thereby producing a plurality of stored times in said memory.

32. An implantable heart monitoring and stimulating system as claimed in claim 31 comprising a first stimulation member carried by said first lead and a second stimulation member carried by said second lead, and wherein said first and second leads also are connected to said pulse generator and wherein said pulse generator is adapted to stimulate the right ventricle via the first stimulation member and to stimulate the left ventricle via the second stimulation member.

33. An implantable heart monitoring and stimulating system as claimed in claim 12 wherein said first and second leads are also connected to said pulse generator, and wherein said pulse generator is adapted to stimulate the right ventricle via said first sensing member and to stimulate the left ventricle via said second sensing member

34. A method for monitoring and stimulating a heart, comprising the steps of:
a housing adapted for implantation in a subject;
a first lead adapted for implantation in the subject for interacting with the right ventricle of the heart of the subject;
a second lead carrying a second sensing member adapted to interact with the left ventricle of the heart of the subject;
a sensing circuit in said housing connected to said first and second leads for sensing, via said first sensing member, cardiac events related to the right ventricle and for sensing, via said second sensing member, cardiac events related to the left ventricle;
a pulse generator adapted to interact with the right ventricle to electrically stimulate the right ventricle and to interact with the left ventricle to electrically stimulate the left ventricle;
a memory in said housing; and
a control circuit disposed in said housing connected to said sensing circuit for receiving signals representing the sensed cardiac events related to the right ventricle and signals representing sensed cardiac events related to the left ventricle therefrom and connected to said pulse generator for controlling stimulation of the right and left ventricle, and having access to said memory, said control circuit determining a time, within a heart cycle, between a first event related to one of said right and left ventricles and a second event related to the other of said right and left ventricles, and storing said determined time in said memory, and performing a plurality of repetitions of determining and storing said time at a plurality of occasions, thereby producing a plurality of stored times in said memory.

35. A method as claimed in claim 34 comprising periodically transferring said stored times from said memory to an extracorporeal apparatus.

36. A method as claimed in claim 35 comprising producing a graph from the stored times transferred to said extracorporeal apparatus representing a change of a heart condition of said subject.

37. A method as claimed in claim 34 comprising using said graph to monitor a change associated with cardiomyopathy.

38. A method as claimed in claim 17 comprising implanting said housing and said first and second leads in a subject suffering from a bundle branch block.
